(19) United States
(12) Patent Application Publication
(10) Pub. No.: US 2004/0054001 A1
(43) Pub. Date: Mar. 18, 2004

(54) Fumaric acid derivatives as nf-kappab inhibitors

(76) Inventors: Rajendra Kumar Joshi, Zurich (CH); Hans-Peter Strebel, Luzern (CH); Peter Petzelbauer, Wien (AT)

Correspondence Address: SIEBERTH & PATTY, 2924 BRAKLEY DRIVE SUITE A 1, BATON ROUGE, LA, 70816

(21) Appl. No.: 10/250,983

(22) PCT Filed: Jan. 8, 2002
(86) PCT No.: PCT/EP02/00108

(30) Foreign Application Priority Data
Jan. 12, 2001 (DE) 101 01 307.8

(51) Int. Cl. A61K 31/275; A61K 31/255

(52) U.S. Cl. 514/527; 514/540; 514/547

(57) ABSTRACT

The present invention relates to the use of one or more fumaric acid derivatives as NF-kappaB inhibitor. At the same time, the present invention relates to the use of the fumaric acid derivatives for preparing a pharmaceutical composition for treating diseases that may be influenced by NF-kappaB.

The present invention relates to the use of one or more fumaric acid derivatives as NF-kappaB inhibitor. At the same time, the present invention relates to the use of the fumaric acid derivatives for preparing a pharmaceutical composition for treating diseases that may be influenced by NF-kappaB.

It is known that pharmaceutical preparations such as fumaric acid which, upon biological degradation after administration, enter into the citric acid cycle or are part thereof gain increasing therapeutic significance—especially when given in high dosages—since they can alleviate or heal diseases caused cryptogenetically. In addition, fumaric acid inhibits the growth of the Ehrlich ascites tumour in mice, reduces the toxic effects of mitomycin C and aflatoxin and displays anti-psoriatic and anti-microbial activity.

The most important practical application is the treatment of psoriasis with various fumaric acid derivatives which has already been described in a number of patents, for example EP 0 188 479, DE 25 30 372, DE 26 21 214 or EP 0 312 697.

Another use of certain fumaric acid derivatives, namely of the alkyl hydrogen fumarates, is disclosed in DE 197 21 099.6 and DE 198 53 487.6 according to which these specific fumaric acid derivatives are described for treating auto-immune diseases such as polyarthritis, multiple sclerosis and graft-versus-host reaction. In addition, DE 198 53 487.6 and DE 198 39 566.3 teach the use of alkyl hydrogen fumarates and dialkyl fumarates in transplantation medicine. Even though individual investigations of the action mechanism of fumaric acid derivatives in the treatment of psoriasis have been carried out, no specific information exists on this topic.

The NF-kappaB (nuclear factor kappaB) is a transcription factor of eukaryotic cells. NF-kappaB belongs to the family of Rel proteins, a class of transcription factors characterised by a so-called Rel domain. The Rel domain has been named after the first member found in an avian virus as an oncogen. Specific sites of this homologous Rel domain (Rel homology domain=RHD) which consists of 300 amino acids are responsible for the DNA bonding to the kappaB sites, the dimerisation with other proteins of the Rel family and the interaction with I-kappaB.

So far, five members of the Rel family are known in mammals. These are c-Rel, NF-kappaB1 (p105/p50), NF-kappaB2 (p100/p52) and RelB. In theory, these five members of the Rel protein family may combine into any form of homo- and heterodimers, even though only a few specific combinations have been observed in vivo. The classic and best characterised NF-kappaB molecule is a heterodimer of the p50/p65 sub-units NF-kappaB1/RelA. This heterodimer is the most common complex and is found in practically all cell types.

After the cellular activation and the dissociation of 1-kappaB, the NF-kappaB heterodimer p50/p65 migrates into the cell nucleus where it binds to the consensus sequence 5′-GGGRNNYYCC-3′. In this process, the p50 sub-unit primarily serves as the DNA-binding sub-unit, while the p65 sub-unit provides the transactivation function.

As a result of the different combinations, each of these heterodimers displays unique characteristics as far as cell type specificity, preferences with regard to DNA-bonding, differential interaction with 1-kappaB isoforms, differential activation requirements and the kinetics of activation are concerned.

The rapid inducibility of NF-kappaB is attributed to the fact that the factor is present in the cytoplasm in an inactive form, namely in a complex bonded to the NF-kappaB inhibitor I-kappaB. Therefore, no new protein synthesis is required for activation, but merely the solution of this complex with I-kappaB or degradation of this inhibitor and subsequent translocation of the now active NF-kappaB dimer into the nucleus.

NF-kappaB may be activated by a large variety of physiological and non-physiological stimuli. These include cytokines, mitogenes, viruses, viral products, the cross-linking of antigen receptors on T- and B-lymphocytes, calcium ionophores, phorbol esters, UV-rays, oxidation stress, phosphatase inhibitors and others. The range of the many NF-kappaB regulated or activated genes is just as broad, the transcription of which is activated, induced or enhanced by the bonding of the heterodimer to the consensus sequence as described above. Especially TNF-alpha. IL-1, IL-2 and lipopolysaccharides may be mentioned as important stimulants.

The regulated genes generally comprise genes involved in the immune function, inflammation response, cell adhesion, cell growth, but also cell death. Genes of cell adhesion molecules, cytokines, cytokine receptors, acute phase proteins, growth factors and viral genes should be mentioned in particular. Special among the genes induced by NF-kappaB are the genes for interferon-β, for the light chain of the immunoglobulin, for the T-cell receptor, for TNF-α and TNF-β and for the tissue factor (CD142), formerly called tissue tromboplastin or factor III.

Owing to its central role in the regulation of immune reactions and inflammation responses shown above and its involvement in the regulation of tissue factors, cytokines etc. it was assumed that advantages similar to those already known from anti-inflammatory agents may be expected from the development of selective inhibitors for the transcription factor NF-kappaB. Steroidal anti-inflammatory agents, interferons or cyclosporine may be named as examples.

Surprisingly, it has now been found that individual fumaric acid derivatives or mixtures thereof have an NF-kappaB inhibiting effect. This effect may preferably be used for the preparation of a pharmaceutical composition containing these fumaric acid derivatives individually or in admixture for the therapy of diseases that are mediated or may be influenced by NF-kappaB. In particular, diseases that may be influenced by NF-kappaB are progressive systemic sclerodermia, osteochondritis syphilitica (Wegener's disease), cutis marmorata (livedo reticularis), Behcet disease, panarteriitis, colitis ulcerosa, vasculitis, osteoarthritis, gout, arteriosclerosis, Reiter's disease, pulmonary granulomatosis, types of encephalitis, endotoxic shock (septic-toxic shock), sepsis, pneumonia, encephalomyelitis, anorexia nervosa, hepatitis (acute hepatitis, chronic hepatitis, toxic hepatitis, alcohol-induced hepatitis, viral hepatitis, jaundice, liver insufficiency and cytomegaloviral hepatitis), Rennert T-lymphomatosis, mesangial nephritis, post-angioplastic restenosis, reperfusion syndrome, cytomegaloviral retinopathy, adenoviral diseases such as adenoviral colds, adenoviral pharyngoconjunctival fever and adenoviral ophthalmia, AIDS, Guillain-Barre syndrome, post-herpetic or post-zoster neuralgia, inflammatory demyelinising polyneuropathy, mononeuropathia multiplex, mucoviscidosis, Bechterew's disease, Barett oesophagus, EBV (Epstein-Barr virus) infection, cardiac remodeling, interstitial cystitis diabetes mellitus type II, human tumour radiosensitisation, multi-resistance of malignant cells to chemotherapeutic agents (multidrug resistance in chemotherapy), granuloma annulare and cancers such as mamma carcinoma, colon carcinoma, melanoma, primary liver cell carcinoma, adenocarcinoma, kaposi's sarcoma, prostate carcinoma, leukaemia such as acute myeloid leukaemia, multiple myeloma (plasmoctoma), Burkitt lymphoma and Castleman tumour.

According to the invention, one or more fumaric acid derivatives selected from the group consisting of fumaric acid dialkyl esters and fumaric acid monoalkyl esters in the form of the free acid or in the form of salts and mixtures thereof are preferably used for NF-kappaB inhibition and for preparing the pharmaceutical composition.

The fumaric acid dialkyl esters preferably correspond to the formula

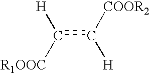

wherein R₁ and R₂, which may be the same or different, independently represent a linear, branched, cyclic, saturated or unsaturated C₁₋₂₄ alkyl radical or a C₅₋₂₀ aryl radical and these radicals are optionally substituted with halogen (F, Cl, Br, I), hydroxy, C₁₋₄ alkoxy, nitro or cyano.

The radicals R₁ and R₂ are preferably methyl, ethyl, n-propyl, isopropyl, n-butyl, sec-butyl, t-butyl, pentyl, cyclopentyl, 2-ethylhexyl, hexyl, cyclohexyl, heptyl, cycloheptyl, octyl, vinyl, allyl, 2-hydroxyethyl, 2- or 3-hydroxypropyl, 2,3-dihydroxypropyl, 2-methoxyethyl, methoxymethyl or 2- or 3-methoxypropyl.

The fumaric acid monoalkyl esters preferably correspond to the formula

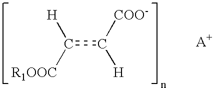

wherein R₁ is as defined above, A is hydrogen, an alkali or alkaline earth metal cation or a physiologically compatible transition metal cation, preferably selected from Li⁺, Na⁺, K⁺, Mg²⁺, Ca²⁺, Zn²⁺, Fe²⁺ and Mn²⁺ and n is 1 or 2 and corresponds to the valence of A

The invention preferably uses one or more fumaric acid derivative(s) selected from the group comprising fumaric acid dimethyl ester, fumaric acid diethyl ester, fumaric acid methylethyl ester, methyl hydrogen fumarate, ethyl hydrogen fumarate, magnesium methyl fumarate, magnesium ethyl fumarate, zinc methyl fumarate, zinc ethyl fumarate, iron methyl fumarate, iron ethyl fumarate, calcium methyl fumarate and/or calcium ethyl fumarate.

According to the invention, the fumaric acid derivatives for preparing the pharmaceutical composition are preferably used in such an amount that one dosage unit of said pharmaceutical composition contains an amount of fumaric acid derivative(s) corresponding or equivalent to 1 to 500 mg, preferably 10 to 300 mg and most preferably 10 to 200 mg of fumaric acid.

Preferred forms of administration for the pharmaceutical composition are oral, parenteral, rectal transdermal, dermal, nasal, pulmonal (inhalation) or ophthal administration (in the form of eye drops), oral administration being preferred. The composition will then be present in a suitable form for each type of administration.

When administered orally, the pharmaceutical composition is present in the form of single unit dose tablets, micro-tablets (multiple unit dose tablets) or mini-tablets, micro-pellets or granulate (said micro-tablets, micro-pellets or the granulate optionally being encapsulated or filled into sachets), capsules or solutions for drinking. In a preferred embodiment, solid dosage or administration forms are provided with an enteric coating. Such a coating may also be provided on encapsulated or filled dosage forms.

In case of parenteral administration by injection (i.v., i.m., s.c., i.p.), the composition is present in a suitable form. All customary liquid carriers suitable for injections may be used.

The pharmaceutical composition may preferably contain either individually or in admixture: 10 to 500 mg of dialkyl fumarate, especially dimethyl fumarate and/or diethyl fumarate; 10 to 500 mg of calcium alkyl fumarate, especially calcium methyl fumarate and/or calcium ethyl fumarate; 0 to 250 mg of zinc alkyl fumarate, especially zinc methyl fumarate and/or zinc ethyl fumarate; 0 to 250 mg of alkyl hydrogen fumarate, especially methyl hydrogen fumarate and/or ethyl hydrogen fumarate; and 0 to 250 mg magnesium alkyl fumarate, especially magnesium methyl fumarate and/or magnesium ethyl fumarate, the total of the above-mentioned amounts corresponding to an equivalent of 10 to 500 mg, preferably 10 to 300 mg and most preferably 100 mg of fumaric acid.

Preferred compositions of the invention contain only dimethyl fumarate in an amount of 10 to 300 mg.

According to a particularly preferred embodiment, the composition is present in the form of micro-tablets or micro-pellets. These preferably have a size or mean diameter of ≦5000 μm, more preferably 300 to 2500 μm, especially 300 to 1000 μm for pellets and 1000 to 2500 μm for the micro-tablets. By administering the fumaric acid derivatives in the form of micro-tablets, which is preferred in accordance with the invention, gastro-intestinal irritations or side effects which cannot be ruled out in case of administration of customary single-unit dose tablets may be further reduced. This is probably due to the fact that micro-tablets, preferably micro-tablets with enteric coating, are already dispersed in the stomach and therefore reach the intestinal tract in portions where the active ingredients are released in locally smaller doses while the overall dose remains the same. This, in turn, helps avoid local irritation of the epithelial intestinal cells resulting in improved gastro-intestinal tolerance of the micro-tablets in comparison with conventional tablets.

For example, the fumaric acid derivatives contained in the compositions of the invention are prepared by the process described in EP 0 312 679.

### PRODUCTION EXAMPLES

In principle, the oral compositions of the invention in the form of tablets or micro-tablets may be prepared by classic tabletting procedures. Instead of such classic tabletting procedures other methods for preparing tablets may be used, such as direct tabletting as well as processes for preparing solid dispersions according to the melt process or the spray drying process.

The tablets may be provided with an enteric coating. The enteric coating may be applied in a classic coating pan or sprayed on. The coating may also be applied with a Boegel coating apparatus. In addition, the tablet may be provided with a film coat.

In order to explain the use according to the invention, various examples for preparing preferred drugs are given below. These examples are intended to illustrate, but not to limit the invention.

### Example 1

Preparation of Film Tablets with an Enteric Coating Containing 100.0 mg of Monomethyl Fumarate-Ca Salt, which Corresponds to 78 mg of Fumaric Acid

Taking the necessary precautions (breathing mask, gloves, protective clothing, etc.), 10 kg of monomethyl fumarate-Ca salt are crushed, mixed intensely and homogenised by means of a sieve 800. Then an excipient mixture of the following composition is prepared: 21 kg of starch derivative (STA-RX 1500®), 2 kg of micro-crystalline cellulose (Avicel PH 101®), 0.6 kg of polyvinyl pyrrolidone (PVP, Kollidon® 25), 4 kg of Primogel®, 0.3 kg of colloidal silicic acid (Aerosil®).

The active ingredient is added to the entire powder mixture, mixed, homogenised by means of a sieve 200 and processed with a 2% aqueous solution of polyvinyl pyrrolidone (PVP, Kollidon® 25) in the usual manner into binder granules, and then mixed with the outer phase in a dry state. The latter consists of 2 kg of a so-called FST complex containing 80% of talcum, 10% of silicic acid and 10% of magnesium stearate.

Thereafter the mixture is pressed into convex tablets with a weight of 400 mg and a diameter of 10.0 mm by the usual method. Instead of these classic compaction methods, other methods such as direct compaction or solid dispersions according to the melting and spray drying method may also be used for preparing tablets.

Enteric Coating:

A solution of 2.250 kg of hydroxy propyl methyl cellulose phthalate (HPMCP, Pharmacoat HP® 50) is dissolved in a solvent mixture consisting of 2.50 litres of demineralised water. 13 litres of acetone Ph. Helv. VII and 13 litres of ethanol (94% by weight) and then 0.240 kg of castor oil (Ph. Eur. II) is added to the solution. The solution is poured or sprayed in portions onto the tablet cores in a coating pan in a conventional manner or applied by means of a fluidised-bed apparatus of the appropriate structure.

After drying, the film coating is applied. Said coating consists of a solution of Eudragit E 12.5%® 4.8 kg, talcum Ph. Eur. II 0.34 kg, titanium(VI) oxide Cronus RN 56® 0.52 kg, coloured lacquer ZLT-2blue (Siegle) 0.21 kg, and polyethylene glycol 6000 Ph. Helv. VII 0.12 kg in a solvent mixture of 8.2 kg of 2-propanol Ph. Helv. VII, 0.06 kg of glycerine triacetate (Triacetin®) and 0.2 kg of demineralised water. After homogenous distribution in the coating pan or the fluidised bed, the mixture is dried and polished in the usual manner.

### Example 2

Preparation of Enteric Coated Capsules Containing 86.5 mg of Monoethyl Fumarate-Ca Salt and 110.0 mg of Dimethyl Fumarate, which Corresponds to a Total of 150 mg of Fumaric Acid

Taking the necessary precautions (breathing mask, gloves, protective clothing, etc.), 8.65 kg of monoethyl fumarate-Ca salt and 11 kg of dimethyl fumarate are intensely mixed with a mixture consisting of 15 kg of starch, 6 kg of lactose Ph. Helv. VII, 2 kg of micro-crystalline cellulose (Avicel®), 1 kg of polyvinyl pyrrolidone (Kollidon® 25) and 4 kg of Primogel® and homogenised by means of a sieve 800.

Together with a 2% aqueous solution of polyvinyl pyrrolidone (Kollidon® 25) the entire powder mixture is processed in the usual manner into a binder granulate and mixed with the outer phase in the dried state. Said outer phase consists of 0.35 kg of colloidal silicic acid (Aerosil®), 0.5 kg of Mg stearate and 1.5 kg of talcum Ph. Helv. VII. The homogeneous mixture is then filled in portions of 500.0 mg into appropriate capsules which are then provided with an enteric (gastric-acid resistant) coating consisting of hydroxy propyl ethyl cellulose stearate and castor oil as softening agent by a known method. Instead of hard gelatine capsules, the mixture may also be filled into appropriate gastric acid-resistant capsules, which consist of a mixture of cellulose acetate phthalate (CAP) and hydroxy propyl ethyl cellulose phthalate (HPMCP).

### Example 3

Preparation of Enteric-Coated Micro-Tablets in Capsules Containing 87.0 mg of Monoethyl Fumarate-Ca Salt, 120 mg of Dimethyl Fumarate, 5.0 mg of Monoethyl Fumarate-Mg Salt and 3.0 mg of Monoethyl Fumarate-Zn Salt, which Corresponds to a Total of 164 mg of Fumaric Acid (“forte” Tablets)

Taking the necessary precautions (breathing mask, gloves, protective clothing, etc.), 8.7 kg of monoethyl fumarate-Ca salt, 12 kg of dimethyl fumarate, 0.5 kg of monoethyl fumarate-Mg salt and 0.3 kg of monoethyl fumarate-Zn salt are crushed, intensely mixed and homogenised by means of an sieve 800. Then an excipient mixture of the following composition is prepared: 18 kg of starch derivative (STA-RX 1500), 0.3 kg of micro-crystalline cellulose (Avicel PH 101), 0.75 kg of PVP (Kollidon 120), 4 kg of Primogel, 0.25 kg of colloidal silicic acid (Aerosil). The entire powder mixture is added to the active ingredient mixture, homogenised by means of a 200 sieve, processed in the usual manner with a 2% aqueous solution of polyvinyl pyrrolidone (Kollidon K25) to obtain a binder granulate and mixed in a dry state with the outer phase consisting of 0.5 kg of magnesium stearate and 1.5 kg of talcum. Then the powder mixture is pressed by the conventional method into convex micro-tablets with a gross mass of 10.0 mg and a diameter of 2.0 mm. Instead of this classic tabletting method other methods for making tablets such as direct tabletting or solid dispersions by the melt method and the spray drying method may also be used.

The gastric acid-resistant coating may be poured or sprayed on in a classic coating pan or applied in a fluidised-bed apparatus. In order to achieve resistance to gastric acid, portions of a solution of 2.250 kg of hydroxy propyl methyl cellulose phthalate (HPMCP, Pharmacoat HP 50) are dissolved in a mixture of the following solvents: acetone 13 l, ethanol 94% by weight denatured with 2% ketone 13.5 l and demineralised water 2.5 l. 0.240 kg of castor oil are added as softening agent to the finished solution and applied in portions to the tablet cores in the usual manner.

Film-coat: After drying is completed, a suspension of the following composition is applied as a film-coat in the same apparatus: talcum 0.340 kg, titanium(VI) oxide Cronus RN 56 0.4 kg, coloured lacquer L red lacquer 86837 0.324 kg, Eudragit E 12.5% 4.8 kg and polyethylene glycol 6000 pH 11 XI 0.12 kg in a solvent mixture of the following composition: 2-propanol 8.17 kg, aqua demineralisata 0.2 kg and glycerine triacetate (Triacetin) 0.6 kg.

The gastric acid-resistant micro-tablets are then filled into hard gelatine capsules at a net weight of 500.0 mg and sealed.

### Example 4

Preparation of Enteric-Coated Micro-Tablets in Capsules Containing 120.0 mg of Dimethyl Fumarate which Corresponds to 96 mg of Fumaric Acid

Taking the necessary precautions (breathing mask, gloves, protective clothing, etc.), 12 kg of dimethyl fumarate are crushed and homogenised by means of a sieve 800. Then an excipient mixture of the following composition is prepared: 17.5 kg of starch derivative (STA-KX 1500®), 0.30 kg of micro-crystalline cellulose (Avicel PH 101®), 0.75 kg of PVP (Kollidon® 120), 4 kg of Primogel®, 0.25 kg of colloidal silicic acid (Aerosil®). The active ingredient is added to the entire powder mixture, mixed, homogenised by means of a sieve 200 and processed with a 2% aqueous solution of polyvinyl pyrrolidone (Kollidon® 25) in the usual manner into binder granules, and then mixed with the outer phase in a dry state. The latter consists of 0.5 kg of magnesium stearate and 1.5 kg of talcum.

Thereafter the powder mixture is pressed into convex tablets having a gross weight of 10.0 mm and a diameter of 2.0 mm by the usual method.

To achieve resistance against gastric fluid, a solution of 2.25 kg of hydroxy propyl methyl cellulose phthalate (HPMCP, Pharmacoat HP®) 50) is dissolved in a mixture of the following solvents: 13 litres of acetone, 13.5 litres of ethanol (94% by weight denatured with 2% of ketone) and 1.5 l of aqua demineralisata. Then castor oil (0.24 kg) is added to the finished solution as a softening agent and applied onto the tablet cores in the usual manner.

After drying a suspension of the following composition is applied in the same apparatus as a film coat: talcum 0.34 kg, titanium(VI) oxide Cronus RN 56® 0.4 kg, coloured lacquer L-red 86837 0.324 kg, Eudragit E 12.5%® 4.8 kg and polyethylene glycol 6000 pH 11 XI 0.12 kg in a solvent mixture of the following composition: 8.17 kg of 2-propanol, 0.2 kg of demineralised water and 0.6 kg of glyc-crine triacetate (Triacetin®).

The enteric-coated micro-tablets are then filled into hard gelatine capsules at a net weight of 400 mg and sealed.

### Example 5

Preparation of Enteric-Coated Micro-Tablets in Capsules Containing 120.0 mg of Dimethyl Fumarate, which Corresponds to 96 mg of Fumaric Acid

12 kg of dimethyl fumarate are crushed and homogenised as described above. Then an excipient mixture of the following composition is prepared: 23.2 kg of micro-crystalline cellulose (Avicel PH 200®), 3 kg of croscarmelose sodium (AC-Si-SOL-SD-711), 2.5 kg of talcum, 0.1 kg of anhydrous silicic acid (Aerosil® 200) and 1 kg of Mg stearate. Thereafter the powder mixture is pressed into convex tablets having a gross weight of 10.0 mm and a diameter of 2.0 mm by the usual method.

After that, a solution of 0.94 kg Eudragit® L in isopropanol is prepared which additionally contains 0.07 kg of dibutyl phthalate. This solution is sprayed onto the tablet cores. Then a dispersion of 17.2 kg of Eudragit® L D-55 and a mixture of 2.8 kg of micro-talcum, 2 kg of Macrogol 6000 and 0.07 kg of dimeticon in water is prepared and sprayed onto the cores.

Thereafter, the enteric-coated micro-tablets are filled into hard gelatine capsules at a net weight of 650 mg and sealed.

### Example 6

NF-kappaB Translocation into the Cell Nucleus

NF-kappaB (p65) was inserted into the vector pEGFP-C1 which contained EGFP (green fluorescent protein) linked with a cytomegalovirus promoter (Clontech). This leads to the expression of a fluorescent NF-kappaB. HUVEC cells were plated between the third and the fifth passage in gelatine-coated culture plates having 12 wells (Costar) and grown to 80 or 90% confluence, respectively. Then these cells were subjected to transfection using the calcium phosphate precipitation method. Specifically the cells were conditioned with Dulbecco's modified Eagles medium (DMEM), the precipitate containing 1 μg of DNA per well added after 24 hours and the cells incubated a further four hours. After washing with HBSS (Hanks balanced salt solution), culture medium was added and the cells grown for a further 18 hours before they were stimulated.

For the experiments, the cells were conditioned with 40 μM/l of dimethyl fumarate, parallel preparations without DNA acting as control. 2 hours after commencement of conditioning the cells were stimulated with 10 ng/ml TNF-α for the time stated in table 1.

After that, the cells were subjected to lysis, the supernatant discarded and the cell nuclei collected in Dounce buffer with protease inhibitor (10 mM tris-HCl, pH 7.6, 0.5 mM MgCl, 10 μg/ml leupeptin, 10 μg/ml aprotinin, 1 mM phenyl methyl sulfonyl fluoride, 1.8 mg/ml iodoacetamide). After 10 minutes of centrifugation at 1200 g, 4° C., the cell nuclei were analysed on an FACscanflow cytometer (Becton Dickinson).

TABLE 1
Number of NF-kappaB (p65) positive nuclei
(percentage based on all cells subjected to transfection with NF-kappaB)

| Duration of stimulation | Control | DMF (40 μM/l, n = 3) |
|---|---|---|
| 0 min. | 30 ± 3 | 29 ± 5 |
| 10 min. | 61 ± 5 | 20 ± 4 |
| 30 min. | 50 ± 6 | 25 ± 6 |
| 60 min. | 55 ± 10 | 24 ± 9 |

This table shows that dimethyl fumarate at a concentration of 40 μM/l inhibited the TNF-induced translocation of NF-kappaB into the cell nucleus.

### Example 7

Inhibition of the NF-kappaB Stimulated Transcription

A triple repeat of the ALP-1 consensus site (bonding site) (48 bp, 3×TGTGATGACTCAGGTT) and a triple repeat of the NF-kappaB consensus site (60 bp. 3×AATCGTGGAATTTCCTCTGA), flanked by SpeI bonding sites (not shown) were inserted into the SpeI site of the pTK-UBT-luc vector (de Martin, Gene 124, 137-138, 1993). A 1.3 kb construct of the E-selectin promoter extending from bp −1285 to bp +482 was inserted into the NdeI site of the pMAM Neo-luc vector (Clontech).

HUVEC cells were subjected to transfection with the constructs thus obtained as described in example 6. For said transfection, 2.5 μg of the pertinent promoter construct per well were added. In order to verify the transfection efficiency, co-transfections were carried out with 500 ng of a pSV-beta galactosidase control vector (Promega Corp., Madison, Wis., U.S.A.) as control in each experiment. 2 days after transfection the cells were stimulated for 2 hours with 10 ng/ml TNF-alpha with and without addition of 6 μg/ml dimethyl fumarate (DMF). The cells were then harvested by trypsination, pelletised, washed and re-suspended in 200 μl of “reporter lysis buffer” (Promega) for 15 min. as prescribed by the manufacturer.

The luciferase activity was measured by means of a Berthold AutoLumat LB9507 luminometer using the luciferase test system (Promega). The beta-galactosidase activity was determined using the Promega beta-galactosidase enzyme test system. The luciferase activities obtained with the pertinent promoter constructs were normalised to the beta-galactosidase activity. The variation width of the beta-galactosidase activity within the individual experiments was below 10%. Table 2 shows the individual results x-fold vis-à-vis the base line.

TABLE 2
Increase of transcription
Relative increase of luciferase activity
(measured as x-fold increase vis-à-vis the base line)
after TNF stimulation (10 ng/ml)
with or without 40 μM/l dimethyl fumarate (DMF), n = 6

| Conditions | NF-kappaB | AP-1 |
|---|---|---|
| TNF | 2 ± 3 | 2.2 ± 0.5 |
| TNF + DMF | 2 ± 1 | 2 ± 0.1 |

Table 2 shows that dimethyl fumarate inhibited the TNF induced transcription of a NF-kappaB dependent gene, but not the transcription of an AP-1 dependent gene. Therefore the dimethyl fumarate inhibition is NF-kappaB-specific.

1. The use of one or more fumaric acid derivatives for preparing a pharmaceutical composition for the treatment of diseases that may be influenced by NF-kappaB.

2. The use according to claim 1, characterised in that the fumaric acid derivative is selected from the group consisting of fumaric acid dialkyl esters and fumaric acid monoalkyl esters, which may optionally be substituted, in the form of the free acid or its salts and mixtures thereof.

3. The use according to claim 2, wherein the fumaric acid dialkyl ester corresponds to the formula 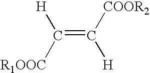
wherein R₁ and R₂, which may be the same or different, independently represent a linear, branched, cyclic, saturated or unsaturated C₁₋₂₄ alkyl radical or a C₅₋₂₀ aryl radical and these radicals are optionally substituted with halogen (F, Cl, Br, I), hydroxy, C₁₋₄ alkoxy, nitro or cyano.

4. The use according to one of the claims 2 and 3, characterised in that the radicals R₁ and R₂ are methyl, ethyl, n-propyl, isopropyl, n-butyl, sec-butyl, t-butyl, pentyl, cyclopentyl, 2-ethylhexyl, hexyl, cyclohexyl, heptyl, cycloheptyl, octyl, vinyl, allyl, 2-hydroxyethyl, 2- or 3-hydroxypropyl, 2,3-dihydroxypropyl, 2-methoxyethyl, methoxymethyl or 2- or 3-methoxypropyl.

5. The use according to claim 2 wherein the fumaric acid monoalkyl ester corresponds to the formula 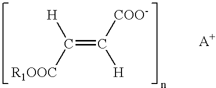
wherein
R₁ is as defined in claims 3 or 4
A is hydrogen, an alkali or alkaline earth metal cation or a physiologically compatible transition metal cation, preferably selected from Li⁺, Na⁺, K⁺, Mg²⁺, Ca²⁺, Zn²⁺, Fe²⁺ and Mn²⁺, and
n is 1 or 2 and corresponds to the valence of A

6. The use according to any of the previous claims, characterised in that the fumaric acid derivative is one or more selected from the group comprising fumaric acid dimethyl ester, fumaric acid diethyl ester, fumaric acid methylethyl ester, methyl hydrogen fumarate, ethyl hydrogen fumarate, calcium methyl fumarate, calcium ethyl fumarate, magnesium methyl fumarate, magnesium ethyl fumarate, zinc methyl fumarate, zinc ethyl fumarate, iron methyl fumarate and iron ethyl fumarate and mixtures thereof.

7. The use according to claim 6, characterised in that the fumaric acid derivative is fumaric acid dimethyl ester (dimethyl fumarate).

8. The use of one or more fumaric acid derivatives for preparing a pharmaceutical composition for the therapy of diseases that may be influenced by NP-kappaB, selected from the group comprising:
progressive systemic sclerodermia, osteochondritis syphilitica (Wegener's disease), cutis marmorata (livedo reticularis), Behcet disease, panarteriitis, colitis ulcerosa, vasculitis, osteoarthritis, gout, arteriosclerosis, Reiter's disease, pulmonary granulomatosis, types of encephalitis, endotoxic shock (septic-toxic shock), sepsis, pneumonia, encephalomyelitis, anorexia nervosa, hepatitis (acute hepatitis, chronic hepatitis, toxic hepatitis, alcohol-induced hepatitis, viral hepatitis, jaundice, liver insufficiency and cytomegaloviral hepatitis), Rennert T-lymphomatosis, mesangial nephritis, post-angioplastic restenosis, reperfusion syndrome, cytomegaloviral retinopathy, adenoviral diseases such as adenoviral colds, adenoviral pharyngoconjunctival fever and adenoviral ophthalmia, AIDS, Guillain-Barré syndrome, post-herpetic or post-zoster neuralgia, inflammatory demyelinising polyneuropathy, mononeuropathia multiplex, mucoviscidosis. Bechterew's disease, Barett oesophagus, EBV (Epstein-Barr virus) infection, cardiac remodeling, interstitial cystitis, diabetes mellitus type II, human tumour radiosensitisation, multi-resistance of malignant cells to chemotherapeutic agents (multidrug resistance in chemotherapy), granuloma annulare and cancers such as mamma carcinoma, colon carcinoma, melanoma, primary liver cell carcinoma, adenocarcinoma, kaposi's sarcoma, prostate carcinoma, leukaemia such as acute myeloid leukaemia, multiple myeloma (plasmocytoma), Burkitt lymphoma and Castleman tumour.

9. The use according to claim 8, characterised in that the fumaric acid derivative is selected from the group consisting of fumaric acid dialkyl esters and fumaric acid monoalkyl esters in the form of the free acid or a salt or mixtures thereof.

10. The use according to claim 9, where the fumaric acid dialkyl ester corresponds to the formula 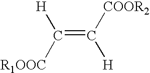
wherein R₁ and R₂, which may be the same or different, independently represent a linear, branched, cyclic, saturated or unsaturated C₁₋₂₄ alkyl radical or a C₅₋₂₀ aryl radical and these radicals are optionally substituted with halogen (F, Cl, Br, I), hydroxy, C₁₋₄ alkoxy, nitro or cyano.

11. The use according to one of the claims 9 and 10, characterised in that the radicals R₁ and R₂ are methyl, ethyl, n-propyl, isopropyl, n-butyl, sec-butyl, t-butyl, pentyl, cyclopentyl, 2-ethylhexyl, hexyl, cyclohexyl, heptyl, cycloheptyl, octyl, vinyl, allyl, 2-hydroxyethyl, 9- or 3-hydroxypropyl, 2,3-dihydroxypropyl, 2-methoxyethyl, methoxymethyl or 2- or 3-methoxypropyl.

12. The use according to claim 9 wherein the fumaric acid monoalkyl ester corresponds to the formula 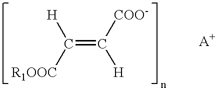
wherein
R₁ is as defined in claims 3 or 4
A is hydrogen, an alkali or alkaline earth metal cation or a physiologically compatible transition metal cation, preferably selected from Li⁺, Na⁺, K⁺, Mg²⁺, Ca²⁺, Zn²⁺, Fe²⁺ and Mn²⁺, and
n is 1 or 2 and corresponds to the valence of A

13. The use according to any of the claims 8 to 12, characterised in that one dosage unit of the pharmaceutical composition contains an amount of fumaric acid derivative(s) corresponding to 1 to 500 mg, preferably 10 to 300 mg and most preferably 10 to 200 mg of fumaric acid.

14. The use according to any of the previous claims 8 to 13 for preparing a pharmaceutical composition for oral, parenteral, rectal, transdermal, dermal, nasal, pulmonal (inhalation) or ophthal administration, preferably for oral administration.

15. The use according to claim 14 where the pharmaceutical composition for oral administration is present in the form of unit dose tablets, micro-tablets, micro-pellets or granulate (said micro-tablets, micro-pellets or the granulate optionally being encapsulated or filled into sachets), capsules or solutions for drinking.

16. The use according to claim 15, characterised in that the solid dosage forms are provided with an enteric coating.

17. The use according to claim 8, characterised in that the dosage units of the pharmaceutical composition preferably contain either individually or in admixture:
10 to 500 mg of dialkyl fumarate, especially dimethyl fumarate and/or diethyl fumarate;
10 to 500 mg of calcium alkyl fumarate, especially calcium methyl fumarate and/or calcium ethyl fumarate
0 to 250 mg of zinc alkyl fumarate, especially zinc methyl fumarate and/or zinc ethyl fumarate,
0 to 250 mg of alkyl hydrogen fumarate, especially methyl hydrogen fumarate and/or ethyl hydrogen fumarate; and
0 to 250 mg magnesium alkyl fumarate, especially magnesium methyl fumarate and/or magnesium ethyl fumarate
the total of the above-mentioned amounts corresponding to an equivalent of 10 to 500 mg, preferably 10 to 300 mg and most preferably 100 mg of fumaric acid.

18. The use according to one of the claims 15 or 16, characterised in that the composition is present in the form of micro-tablets or micro-pellets having a size of ≦5000 μm, preferably a size of 300 to 1000 μm for the pellets and 1000 to 2500 μm for the micro-tablets.
